(19) United States
(12) Patent Application Publication
(10) Pub. No.: US 2008/0281232 A1
(43) Pub. Date: Nov. 13, 2008

(54) Device for Measuring the Diameter of an Aortic Annulus

(75) Inventors: Emmanuel Lansac, Paris (FR); Christian Krauss, Cesson (FR); Isabelle Dicenta, Paris (FR)

Correspondence Address: YOUNG & THOMPSON, 209 Madison Street, Suite 500, ALEXANDRIA, VA, 22314, US

(73) Assignee: ASSISTANCE PUBLIQUE - HOSPAUX DE PARIS, Paris (FR)

(21) Appl. No.: 11/886,332

(22) PCT Filed: Mar. 9, 2006
(86) PCT No.: PCT/FR2006/000530

§ 371 Date: Apr. 2, 2008

(30) Foreign Application Priority Data
Mar. 14, 2005 (FR) 0502496

(51) Int. Cl. A61B 5/00

(52) U.S. Cl. 600/587

(57) ABSTRACT

The invention relates to a device for measuring the diameter of an aortic annulus. The distal end of the device is equipped with an expandable cylindrical measuring head (1) which is designed to be introduced through the aortotomy into the outflow tract of the left ventricle of a heart, while the proximal end of the device is equipped with a means (5) for controlling and measuring the constant, adjustable measurement of the expansion of the head, said measuring head and control and measuring means being connected by a rod (4).

The present invention relates to a device for measuring the diameter of an aortic annulus and the internal diameter of vascular structures and the use thereof.

During some operations on the aortic root, such as positioning a prosthetic aortic annulus, it is necessary to measure the diameter of the natural aortic annulus.

Currently, such a measurement is carried out on a stopped heart whose aortic root is not being charged, using a set of Hegar bougies (calibrating cylinder of 2 mm by 2 mm). That instrument is introduced via an aortotomy and lowered into the outflow chamber of the left ventricle. The use of those Hegar bougies provides subjective information relating to the diameter of the aortic annulus of the “fits/doesn't fit” type. In accordance with the calibre of the bougie, information relating to the “correct” or “incorrect” diameter is obtained. However, taking into consideration the extensible nature of the aortic annulus, a bougie having a calibre of from 2 to 4 mm greater than that of the aortic annulus measured may be introduced readily into the aortic root. This results in quite approximate calibration of the aortic annulus.

An object of the present invention is to overcome this disadvantage by providing a means for measuring the internal diameter of the natural aortic annulus on a stopped heart, which means is accurately representative of the diameter of the natural aortic annulus of an aortic root in a beating heart.

To that end, the invention relates to a device for measuring an aortic annulus comprising, at its distal end, an expansible cylindrical measuring head which is adapted in order to be introduced via aortotomy into the outflow chamber of a left ventricle and, at its proximal end, a means for controlling and measuring the expansion of the head with a constant and adjustable measuring force, and a connecting rod between the measuring head and the control and connection means.

The end of the measuring head is non-invasive and the measuring head has a length which is preferably greater than 10 cm in order to be adapted to the height of the outflow chamber of the left ventricle and the aortic root.

The length of the connecting rod between the measuring head and the control and measurement mechanism is preferably greater than 8 cm in order to allow manipulation of the measuring device by the surgeon from outside the thorax.

The measuring device is preferably adapted in order to measure a diameter of at least between 18 mm and 32 mm, with a measuring force of at least between 3 N and 10 N.

The means for controlling and measuring the expansion of the head is, for example, a micrometric screw with a torque limiter.

The measuring head may comprise three blades which are elongate in a manner parallel with the longitudinal axis of the device and which are radially movable relative to the longitudinal axis of the device between a retracted position and an expanded position, or may comprise a band which is radially expansible relative to the longitudinal axis of the device.

The measuring device is preferably constituted by materials which are suitable for use as a surgical instrument.

The invention also relates to a method for measuring a natural aortic annulus in a stopped heart, with extra-corporal circulation and being accessible via an aortotomy, according to which, via the aortotomy, there is introduced into the outflow chamber of the left ventricle a measuring device according to the invention whose measuring head is retracted and, using the control and measurement means, the measuring head is expanded until the limit measuring force is reached, then the diameter indicated by the control and measurement means is recorded and, after the blades of the measuring head have been retracted, the measuring device is withdrawn.

The invention will now be described in greater detail but in a non-limiting manner with reference to the appended Figures, in which:

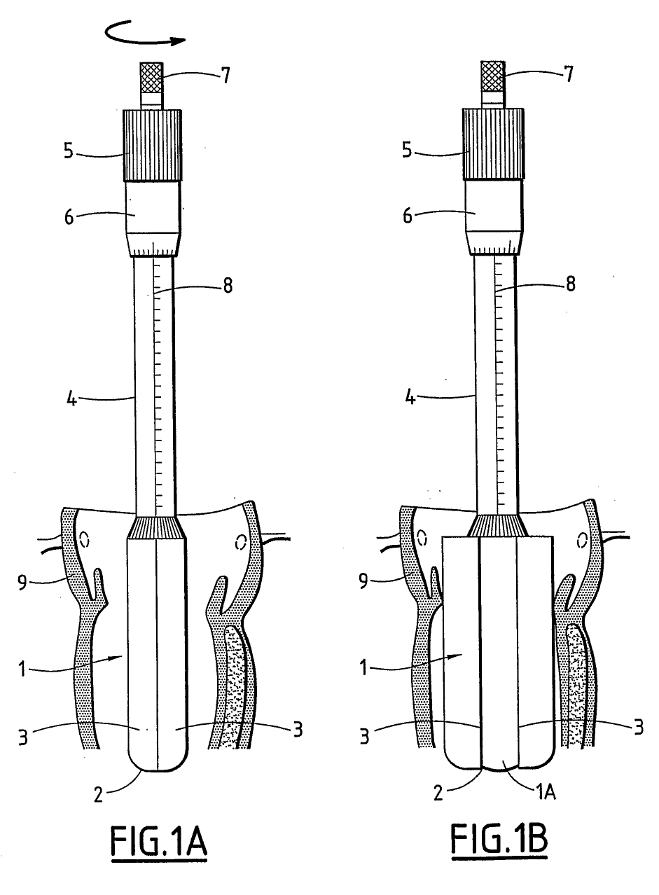

FIGS. 1A and 1B are schematic views of a first embodiment of a device for measuring a natural aortic annulus, arranged in that annulus, firstly in a retracted position and secondly in an expanded position; and

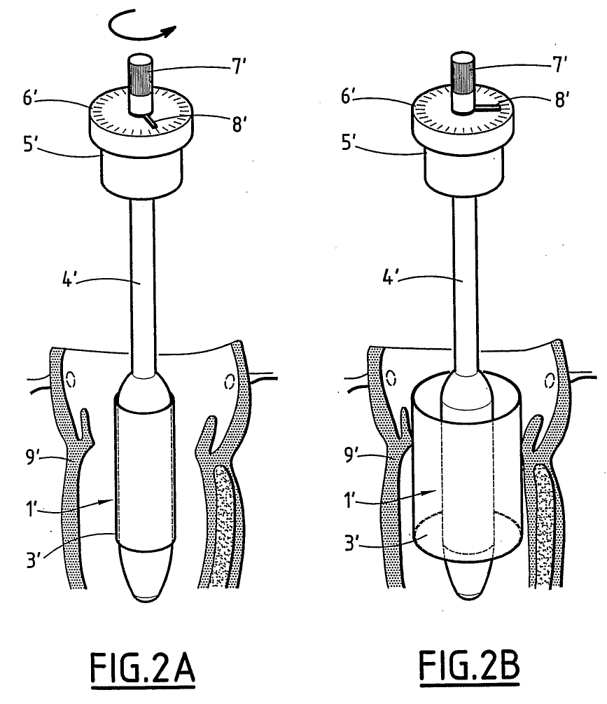

FIG. 2 is a schematic illustration of a second embodiment of a device for measuring a natural aortic annulus, arranged in an aortic annulus, firstly in a retracted position and secondly in an expanded position.

In a first embodiment illustrated in FIGS. 1A and 1B, the device for measuring the natural aortic annulus comprises a measuring head which is generally designated 1 and which is of elongate cylindrical shape having a rounded and soft end 2 so as to be non-invasive. That measuring head is constituted by three blades 3 (only two are visible in FIG. 1A) which extend longitudinally relative to the longitudinal axis of the device and which are radially movable between a retracted position, illustrated in FIG. 1A, and an expanded position, illustrated in FIG. 1B.

The blades are mounted on a mechanism 1A (not visible in detail in the figure) which is known to the person skilled in the art and which allows, by acting on a control means, the blades to be radially displaced, with the displacement of those blades being measured. In particular, the mechanism may be identical to the mechanisms which are known per se from devices for measuring the diameter of a hole, used in mechanical engineering. The measuring head, in its retracted position, has a diameter which is substantially less than or equal to 19 mm and, preferably, 18 mm. In the expanded position, the measuring head has a diameter which is greater than or equal to 31 and, preferably, 32 mm.

The length of the measuring head is adapted so as to be compatible with a good measurement of the diameter of a natural aortic annulus. That length, which is greater than 10 cm, is preferably approximately 15 cm, allows linear contact between the measuring head and the internal wall of the aortic annulus and, consequently, brings about self-centring of the measuring device.

The measuring head, generally designated 1, is carried by a rod 4, at the end of which there is arranged a device 5 for controlling the measuring head, measurement and displaying the measurement of the diameter of the measuring head. Thus, the measuring device comprises, at the distal end thereof, a measuring head and, at the proximal end thereof, a device for controlling and measuring or displaying the measurement.

The rod 4 has a length which is suitable for being able to introduce the measuring device inside the thorax of the patient. That length is at least 10 cm.

The rod 4 may be hollow and contain a shaft (not visible in the figure) allowing the mechanism 1A for expanding the measuring head 1 by means of the control device 5 to be conveyed.

The control, measurement and display device 5 is a device of the torque limiting micrometric screw type, which is known per se and which comprises a drum 6 and a button 7. The torque limiting mechanism is known per se and may be of the type involving a ratchet type button, ratchet type movable drum or friction type movable drum. It allows a measurement to be carried out with constant effort, that is to say, a measurement for which the maximum force applied by the blades to the walls of the aortic annulus is constant and limited. That measuring force may be adjusted by the devices which are known per se to the person skilled in the art and which have been set out above. Preferably, however, they are adjusted to a value of between 3 N and 10 N and preferably between 3.5 N and 9 N in order to allow measurements of the aortic annulus to be carried out corresponding to the mean diastolic pressure of the aortic root of a beating heart, approximately 80 mm of mercury.

The device 5 which is associated with the rod 4 comprises graduations 8 which serve to display the measurement.

This apparatus thereby allows the diameter of a natural aortic annulus 9 to be measured under conditions which are similar to normal physiological conditions with a precision in the order of 1 mm.

In a second embodiment indicated in FIGS. 2A and 2B, in which the same references have been used as in the preceding Figures, but with primes being inserted, the measuring head 1′, which has a size and shape comparable to those of the measuring head of the preceding case, both with regard to the length and diameter, comprises a non-invasive end 2′ and, at its periphery, a measuring means which is constituted by an expansible band 3′. That band is expansible radially by means of a mechanism which is known to the person skilled in the art.

The measuring head, which is adapted in order to be introduced into a natural aortic annulus 9′, is located at the distal end of the apparatus and is carried by a rod 4′ whose length, as in the preceding case, is at least 10 cm and at the end of which there is arranged a means 5′ for controlling the measuring head and displaying the measurement.

This control and display means is located at the proximal end of the device.

In the embodiment illustrated in FIG. 2A or 2B, the control and display means 5′ comprises a graduated disc 6′ and a control piston 71 which is intended to vary the diameter of the measuring band 3′ and which comprises a needle 8′ which indicates the diameter defined by the measuring band 3′. The device further comprises a force limiter, for example, by means of friction, so that, when the band 3′ abuts against the walls of a natural aortic annulus 9′ and the force reaches a value which is defined in advance, the movement of the band 3′ is stopped even if the operator continues to apply a force to the piston 7′.

With this device, the measurement is carried out under the same conditions as with the device which was previously described.

In greater detail, when a surgeon wishes to measure the diameter of a natural aortic annulus, he introduces the measuring device which has been described above, in the retracted state, via the opening of the thorax and the aortotomy, into the outflow chamber of the left ventricle of a stopped heart with extra-corporal circulation.

When the measuring head is correctly positioned inside the natural aortic annulus, by means of the control and display device, the surgeon brings about the expansion of the measuring head until it comes into abutment against the natural aortic annulus and it is stopped because the measuring force is reached. When the measuring force is reached, the surgeon reads the diameter of the natural aortic annulus from the display means. He can then retract the measuring device and withdraw it from the body of the patient.

An advantage of carrying out the measurement with a constant measuring force corresponding to a mean diastolic aortic pressure is that it results in a measurement of the diameter similar to normal physiological conditions when the aortic valve is closed and the aortic root is being charged.

It will be appreciated that, in all cases, the measuring head which is elongate has linear contact with the internal wall of the aortic annulus, which ensures self-alignment of the instrument perpendicularly relative to the cross-section of the aortic annulus.

The device which is intended to be used substantially under surgical conditions is constituted by materials which are compatible with its use as a surgical instrument, that is to say, materials which are well-suited to sterilisation and which are biocompatible. These are, for example, stainless steels which are adapted to the manufacture of surgical instruments.

The two embodiments which have been described above are not limiting and any device which uses a radially expansible measuring head having a shape adapted to use in an aortic annulus and which comprises means for applying a limited and constant measuring effort can be used to construct a measuring device according to the invention.

1. Device for measuring the diameter of an aortic annulus comprising, at the distal end thereof, an expansible cylindrical measuring head (1, 1′) which is adapted in order to be introduced via aortotomy into the outflow chamber of a left ventricle, and, at the proximal end thereof, a means (5, 5′) for controlling and measuring the expansion of the head with a constant and adjustable measuring force, and a connecting rod (4, 4′) between the measuring head and the control and measurement means.

2. Device according to claim 1, characterised in that the end (2, 2′) of the measuring head (5, 5′) is non-invasive.

3. Device according to claim 1, characterised in that the measuring head (1, 1′) has a length greater than 10 cm and is adapted for measuring an aortic annulus.

4. Device according to claim 1, characterised in that the length of the connecting rod (4, 4′) between the measuring head (1, 1′) and the control and measurement mechanism (5, 5′) is greater than 8 cm and is adapted to allow measurement from outside the thorax of a patient.

5. Device according to claim 1, characterised in that it is adapted in order to measure a diameter of at least between 18 mm and 32 mm, with a measuring force of at least between 3 N and 10 N.

6. Device according to claim 1, characterised in that the means (5) for controlling and measuring the expansion of the head is a micrometric screw with a torque limiter.

7. Device according to claim 1, characterised in that the measuring head (1) comprises three blades (3) which are elongate in a manner parallel with the longitudinal axis of the device and which are radially movable relative to the longitudinal axis of the device.

8. Device according to claim 1, characterised in that the measuring head (1′) comprises a band (3′) which is radially expansible relative to the longitudinal axis of the device.

9. Device according to claim 1, characterised in that it is constituted by materials which are suitable for use as a surgical instrument.

10. Method for measuring the diameter of a natural aortic annulus of a stopped heart, with extra-corporal circulation and being accessible via an aortotomy, according to which, via the aortotomy, a measuring device according claim 1 whose measuring head is retracted is introduced into the outflow chamber of the left ventricle, and, using the control and measurement means, the measuring head is expanded until a predefined limit measuring force is reached, then the diameter indicated by the control and measurement means is recorded and, after the measuring head has been retracted, the measuring device is withdrawn.

11. Device according to claim 2, characterised in that the measuring head (1, 1′) has a length greater than 10 cm and is adapted for measuring an aortic annulus.

12. Device according to claim 2, characterised in that the length of the connecting rod (4, 4′) between the measuring head (1, 1′) and the control and measurement mechanism (5, 5′) is greater than 8 cm and is adapted to allow measurement from outside the thorax of a patient.

13. Device according to claim 3, characterised in that the length of the connecting rod (4, 4′) between the measuring head (1, 1′) and the control and measurement mechanism (5, 5′) is greater than 8 cm and is adapted to allow measurement from outside the thorax of a patient.
